(19) United States
(12) Patent Application Publication
(10) Pub. No.: US 2007/0249845 A1
(43) Pub. Date: Oct. 25, 2007

(54) Process for preparing a crystalline form of atorvastatin hemi-calcium

(76) Inventor: Michael Pinchasov, Dover, NJ (US)

Correspondence Address: KENYON & KENYON LLP, ONE BROADWAY, NEW YORK, NY, 10004, US

(21) Appl. No.: 11/713,220

(22) Filed: Mar. 1, 2007

Related U.S. Application Data
(60) Provisional application No. 60/778,333, filed on Mar. 1, 2006.

(51) Int. Cl. C07D 207/30

(52) U.S. Cl. 548/517; 548/562

(57) ABSTRACT

The present invention encompasses a process for preparing crystalline atorvastatin hemi-calcium characterized by a powder X-ray diffraction pattern having broad peaks in the range of 18.5-21.8 and 21.8-25.0±0.2 degrees two theta.

### CROSS-REFERENCE TO RELATED APPLICATIONS

This application claims the benefit of U.S. Provisional Application No. 60/778,333, filed Mar. 1, 2006. The contents of which are incorporated herein by reference.

### FIELD OF THE INVENTION

The present invention encompasses a process for preparing a crystalline atorvastatin hemi-calcium and pharmaceutical formulations thereof.

### BACKGROUND OF THE INVENTION

Atorvastatin,([R—(R*, R*)]-2-(4-fluorophenyl)-β,δ-dihydroxy-5-(1-methylethyl) -3-phenyl-4-[(phenylamino)carbonyl]-1H-pyrrole-1-heptanoic acid), depicted in lactone form in formula (I) and its calcium salt of formula (II) are well known in the art, and described inter alia, in U.S. Pat. Nos. 4,681,893, and 5,273,995, which are herein incorporated by reference.

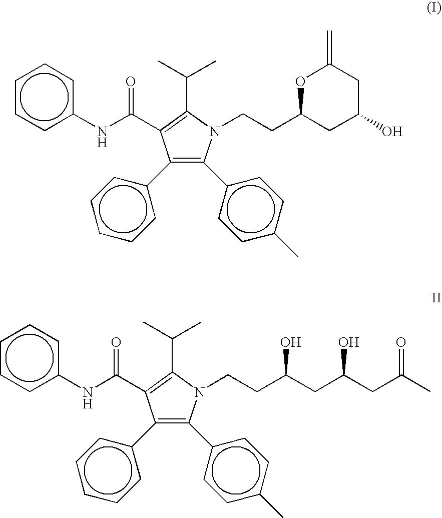

Processes for preparing atorvastatin and its hemi-calcium salt are also disclosed in U.S. publication No. 2002/0099224; U.S. Pat. Nos. 5,273,995; 5,298,627; 5,003,080; 5,097,045; 5,124,482; 5,149,837; 5,216,174; 5,245,047; 5,280,126; Baumann, K.L. et al. Tet. Lett. 1992, 33, 2283-2284, which are hereby incorporated by reference in their entirety and in particular for their teachings related to the preparation of atorvastatin and atorvastatin hemi-calcium.

Atorvastatin is a member of the class of drugs called statins. Statin drugs are currently the most therapeutically effective drugs available for reducing low density lipoprotein (LDL) particle concentration in the blood stream of patients at risk for cardiovascular disease. A high level of LDL in the bloodstream has been linked to the formation of coronary lesions which obstruct the flow of blood and can rupture and promote thrombosis. Goodman and Gilman, The Pharmacological Basis of Therapeutics 879 (9th ed., 1996). Reducing plasma LDL levels has been shown to reduce the risk of clinical events in patients with cardiovascular disease and patients who are free of cardiovascular disease but who have hypercholesterolemia. Scandinavian Simvastatin Survival Study Group, 1994; Lipid Research Clinics Program, 1984a, 1984b.

The mechanism of action of statin drugs has been elucidated in some detail. Statin drugs interfere with the synthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion of HMG to mevalonate, which is the rate determining step in the biosynthesis of cholesterol, and so its inhibition leads to a reduction in the concentration of cholesterol in the liver. Very low density lipoprotein (VLDL) is the biological vehicle for transporting cholesterol and triglycerides from the liver to peripheral cells. VLDL is catabolized in the peripheral cells which releases fatty acids which may be stored in adipocytes or oxidized by muscle. The VLDL is converted to intermediate density lipoprotein (IDL), which is either removed by an LDL receptor, or is converted to LDL. Decreased production of cholesterol leads to an increase in the number of LDL receptors and corresponding reduction in the production of LDL particles by metabolism of IDL.

Atorvastatin hemi-calcium salt trihydrate is marketed under the name LIPITOR® by Pfizer, Inc. Atorvastatin was first disclosed and claimed in U.S. Pat. No. 4,681,893. The hemi-calcium salt depicted in formula (II) is disclosed in U.S. Pat. No. 5,273,995 (“'955 patent”). The '995 patent discloses that the hemi-calcium salt may be obtained by crystallization from a brine solution resulting from the transposition of the sodium salt with CaCl₂ and further purified by recrystallization from a 5:3 mixture of ethyl acetate and hexane.

The occurrence of different crystal forms (polymorphism) is a property of some molecules and molecular complexes. A single molecule, like the atorvastatin in formula (I) or the salt complex of formula (II), may give rise to a variety of solids having distinct physical properties like melting point, X-ray diffraction pattern, infrared absorption fingerprint and NMR spectrum. The differences in the physical properties of polymorphs result from the orientation and intermolecular interactions of adjacent molecules (complexes) in the bulk solid. Accordingly, polymorphs are distinct solids sharing the same molecular formula yet having distinct advantageous and/or disadvantageous physical properties compared to other forms in the polymorph family. One of the most important physical properties of pharmaceutical polymorphs is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. On the other hand, where the effectiveness of a drug correlates with peak bloodstream levels of the drug, a property shared by statin drugs, and provided the drug is rapidly absorbed by the GI system, then a more rapidly dissolving form is likely to exhibit increased effectiveness over a comparable amount of a more slowly dissolving form.

Crystalline Forms I, II, III and IV of atorvastatin hemi-calcium are the subjects of U.S. Pat. Nos. 5,959,156 and 6,121,461, assigned to Warner-Lambert. Crystalline atorvastatin hemi-calcium Form V is disclosed in PCT publication No. WO 01/36384 and is characterized by X-ray powder diffraction peaks at about 5.5 and 8.3±0.2 degrees two-theta and a broad peak at about 18-23 degrees two-theta. The disclosure of Form V and processes for its preparation in WO 01/36384 are incorporated herein by reference. Other crystalline forms of atorvastatin hemi-calcium are disclosed in PCT publication Nos. WO 02/43732 and WO 03/070702.

U.S. Pat. No. 6,605,636 discloses atorvastatin hemi-calcium crystalline form, characterized by a powder X-ray diffraction pattern having broad peaks in the range of 18.5-21.8 and 21.8-25.0±0.2 degrees two theta (therein referred to as Form VII). Form VII is reported to be further characterized by broad peaks at 4.7, 7.8, 9.3, 12.0, 17.1, 18.2±0.2 degrees 2.theta. Examples 1 and 2 of U.S. '636 disclose a method for preparing Form VII by stirring in ethanol.

There is a need in the art for processes which allow for preparation of Form VII that can be used on an industrial scale.

### SUMMARY OF THE INVENTION

The invention encompasses a process for preparing crystalline atorvastatin hemi-calcium comprising: combining crystalline atorvastatin hemi-calcium characterized by X-ray powder diffraction peaks at about 5.5 and 8.3±0.2 degrees two-theta and a broad peak at about 18-23 degrees two-theta and ethanol to obtain a suspension, and spray drying the suspension to obtain crystalline atorvastatin hemi-calcium characterized by a powder X-ray diffraction pattern having broad peaks in the range of 18.5-21.8 and 21.8-25.0±0.2 degrees two theta.

### BRIEF DESCRIPTION OF THE FIGURE

FIG. 1 is an XRD powder pattern of crystalline atorvastatin hemi-calcium Form VII obtained in example 1.

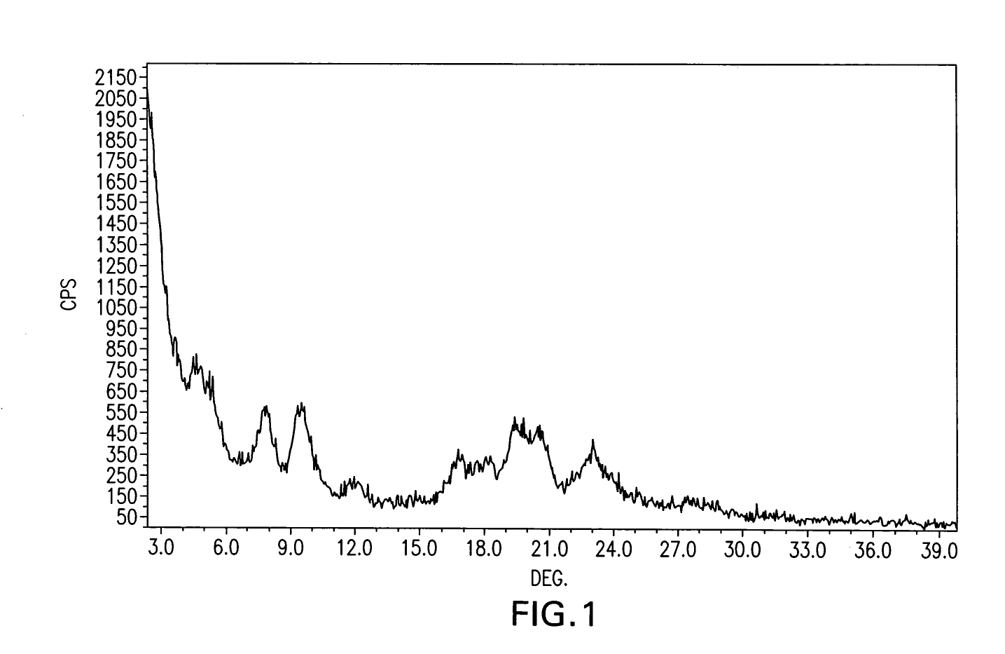

### DETAILED DESCRIPTION OF THE INVENTION

The present invention provides a process for preparing crystalline atorvastatin hemi-calcium characterized by a powder X-ray diffraction pattern having broad peaks in the range of 18.5-21.8 and 21.8-25.0±0.2 degrees two theta (Form VII) suitable for formulation, that can be used on an industrial scale. Specifically, spray drying is used to prepare Form VII. The use of spray drying allows for obtaining a product with high quality suitable for administration to a patient.

The term “spray drying” broadly refers to processes involving breaking up liquid mixtures into small droplets (atomization) and rapidly removing solvent from the mixture. In a typical spray drying apparatus, there is a strong driving force for evaporation of solvent from the droplets, which may be provided by providing a drying gas. Spray drying processes and equipment are described in Perry's Chemical Engineer's Handbook, pgs. 20-54 to 20-57 (Sixth Edition 1984).

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. The process of the invention is not limited to the use of such drying apparatuses as described above.

Spray drying may be performed in a conventional manner in the processes of the present invention (see, e.g., Remington: The Science and Practice of Pharmacy, 19th Ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The atorvastatin hemi-calcium product produced by spray drying may be recovered by techniques commonly used in the art, such as using a cyclone or a filter.

The invention encompasses a process for preparing crystalline atorvastatin hemi-calcium comprising combining crystalline atorvastatin hemi-calcium characterized by X-ray powder diffraction peaks at about 5.5 and 8.3±0.2 degrees two-theta and a broad peak at about 18-23 degrees two-theta (Form V) and ethanol to obtain a suspension, and spray drying the suspension to obtain crystalline atorvastatin hemi-calcium Form VII.

Typically, the suspension is obtained at a temperature of about 10° C. to about 60° C., preferably about 30° C. The suspension is preferably maintained, while stirring, prior to spray drying. Preferably, the suspension is maintained for about 5 to about 64 hours, more preferably for about 17 hours. The concentration of the suspension is preferably about 3% to about 11% of atorvastatin calcium to ethanol by weight.

Typically, spray-drying is performed with a drying gas at an inlet temperature of about 50° C. to about 220° C., more preferably at about 150° C. to about 200° C., most preferably about 200° C. Typically, the outlet temperature of the drying gas is lower than the inlet temperature and is of about 30° C. to about 200° C., preferably about 120° C. to about 130° C.

The drying gas used in the process of the present invention may be any suitable gas, although inert gases such as nitrogen, nitrogen-enriched air, and argon are preferred.

Inlet or outlet temperatures may be varied, if necessary, depending on the equipment, gas, or other experimental parameters. For example, it is known that the outlet temperature may depend on parameters such as aspirator rate, air humidity, inlet temperature, spray air flow, feed rate or concentration.

The spray dried product can be recovered by conventional techniques.

Pharmaceutical compositions for administration to a mammal in need thereof can be prepared from Form VII of the present invention. Such compositions can be prepared by admixing the spray dried Form VII with a pharmaceutically acceptable excipient.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

Powder X-ray diffraction (“PXRD”) analysis was performed using a SCINTAG powder X-ray diffractometer model X'TRA equipped with a solid-state detector. Copper radiation of λ=1.5418 Å was used. The sample was introduced using a round standard aluminum sample holder with round zero background quartz plate in the bottom.

### Example 1

Crystalline atorvastatin hemi-calcium Form V (10 g) was combined with absolute ethanol (300 ml) at about 30° C. to form a mixture. The mixture was stirred for 17 hours. The mixture was then spray dried using a Buchi Mini Spray dryer B-290 with nitrogen drying gas at an inlet temperature of 200° C. and an outlet temperature of 120-130° C. The obtained solid was analyzed by powder X-ray diffraction and determined to be crystalline atorvastatin hemi-calcium Form VII.

What is claimed is:

1. A process for preparing crystalline atorvastatin hemi-calcium characterized by a powder X-ray diffraction pattern having broad peaks in the range of 18.5-21.8 and 21.8-25.0±0.2 degrees two theta comprising combining crystalline atorvastatin hemi-calcium characterized by X-ray powder diffraction peaks at about 5.5 and 8.3±0.2 degrees two-theta and a broad peak at about 18-23 degrees two-theta and ethanol to obtain a suspension, and spray drying the suspension to obtain the crystalline atorvastatin hemi-calcium.

2. The process of claim 1, wherein the suspension is at a temperature of about 10° C. to about 60° C.

3. The process of claim 2, wherein the suspension is at a temperature of about 30° C.

4. The process of claim 1, wherein the suspension is maintained for about 5 to about 64 hours, while stirring, prior to spray drying.

5. The process of claim 4, wherein the suspension is maintained for about 17 hours.

6. The process of claim 1, wherein spray-drying is performed with an inert dry gas at an inlet temperature of about 50° C. to about 220° C.,

7. The process of claim 6, wherein the temperature is about 200° C.

8. The process of claim 1, wherein spray-drying is performed with an inert dry gas at an outlet temperature of about 30° C. to about 200° C.,

9. The process of claim 8, wherein the temperature is about 120° C. to about 130° C.

10. The process of claim 1, wherein the concentration of the suspension is about 3% to about 11% of atorvastatin calcium to ethanol by weight.

11. The process of claim 1, wherein the process is performed on an industrial scale.
